(19) United States
(12) Patent Application Publication
(10) Pub. No.: US 2021/0310948 A1
(43) Pub. Date: Oct. 7, 2021

(54) NANOSTRUCTURES FOR IMPROVED MOLECULAR DETECTION

(71) Applicant: Nicoya Lifesciences, Inc., Kitchener (CA)

(72) Inventors: Ryan Denomme, Kitchener (CA); Sarah STRATHEARN, Waterloo (CA)

(21) Appl. No.: 17/278,621

(22) PCT Filed: Sep. 24, 2019
(86) PCT No.: PCT/IB2019/058104

§ 371 Date: Mar. 22, 2021

Related U.S. Application Data
(60) Provisional application No. 62/735,233, filed on Sep. 24, 2018.

(51) Int. Cl. G01N 21/552; G01N 33/68

(52) U.S. Cl. CPC G01N 21/554; B82Y 5/00; G01N 33/6803

(57) ABSTRACT

Nanostructures for improved molecular detection are disclosed. The nanostructures may be used to increase the ligand immobilization shift to increase the shift in signal of the analyte. In one embodiment, nanostructures are provided that may have small decay lengths and high sensitivity. In another embodiment, nanostructures are provided that may have large decay lengths and 3D surface matrix chemistries.

### CROSS REFERENCE TO RELATED APPLICATIONS

This application is a U.S. National Stage Application of PCT/IB2019/058104 filed on Sep. 24, 2019, which claims benefit of priority to U.S. Provisional Patent Application No. 62/735,233 entitled “NANOSTRUCTURES FOR IMPROVED MOLECULAR DETECTION” filed on Sep. 24, 2018, the entirety of which is incorporated herein by reference.

### TECHNICAL FIELD

The presently disclosed subject matter relates generally to the detection of molecules, such as DNA, proteins, and the like, and more particularly to the analysis of analytes using nanostructures for improved molecular detection.

### BACKGROUND

Localized surface plasmon resonance (LSPR) can be used to detect molecular binding interactions. In LSPR analysis, one molecule, referred to as a ligand, is immobilized onto a nanostructured surface, and another molecule, referred to as an analyte, is passed over the sensor in a microfluidic channel. When the analyte binds to the ligand, the LSPR signal changes. This signal can be quantified as the center position of the LSPR absorbance peak as measured by optical absorbance spectroscopy. The amount that the LSPR peak shifts is dependent on the difference between the analyte refractive index and the background refractive index (i.e., the carrier buffer), as well as the thickness of the analyte layer.

The following equation defines how much the LSPR peak will shift:

$$R = m Δ n ( 1 - {e}^{\frac{- 2 d}{{l}_{d}}} )$$

R=LSPR Peak Position—(change in peak position as a result of the Δn
m=LSPR sensitivity—constant measured in a change in wavelength per unit of refractive index unit (e.g., nm/RIU)
Δn=refractive index change by adding a new layer (i.e., by the analyte binding), can also be expressed as the refractive index of the analyte (n_{analyte}) less a background refractive index (n_{bg}), which may be dominated by the refractive index of a buffer in which the analyte is provided
d=thickness of new layer (of the analyte layer)
l_{d}=decay length (constant)

The refractive index of the analyte can be estimated with the following equation:

$${n}_{analyte} = {n}_{bg} + \frac{{ac}_{s , a} {M}_{analyte}}{{d}_{analyte}} \qquad Equation 1$$

a=an experimentally determined constant depending on the type of analyte, which may be 0.182 g/cm³ for a protein analyte
M_{analyte}=molecular weight of analyte, which may be a protein
d_{analyte}=layer thickness of analyte
c_{s,a}=surface concentration of analyte (this is time dependent and changes throughout the binding event, and is dictated by the surface density of the ligand for surface plasmon resonance (SPR), because it wouldn't be possible to have an analyte binding at a denser surface concentration than the ligand)

Assuming a and c_{s,a} are constant, the amount of peak shift depends on the molecular weight of the analyte and the thickness of the analyte.

Many applications in which binding kinetics are measured include the use of small molecules as the analyte and large molecules like proteins or antibodies as the ligand. A small molecule may be defined as organic molecules that have a molecular weight of less than 1 kDa. Accordingly, the analyte is often a relatively small molecule relative to the ligand. Based on the above equations, a small molecule analyte may not produce a very large change in refractive index when it is binding to a large molecule ligand, thus leading to a difficulty in accurately measuring the amount of change as a result of a binding event. This is because the surface concentration of the small molecule analyte will be relatively low due to the large size of the protein ligand to which it binds, the molecular weight of the small molecule analyte is small, and the thickness added by the small molecule analyte will be very small, if any. Because of the small change in refractive index, the signals produced by small molecule analytes are generally very small and difficult to measure. The amount of shift produced from an analyte can be roughly calculated as follows:

$$Analyte maximum shift = ligand immobilization shift \times \frac{molecular weight of analyte}{molecular weight of ligand} \times number of binding sites$$

Small molecules typically cannot be used as the ligand due to the limited number of functional groups they have and the importance of the availability of functional groups in the interactions to ensure that the measurement of the binding kinetics is not falsely skewed due to saturation of binding with functional groups. To improve the shift in signal of the small molecule, the presently disclosed invention seeks to increase the ligand immobilization shift.

### SUMMARY

A first aspect of the invention includes a surface plasmon resonance (SPR) sensor medium. The medium includes a nanostructure portion comprising a ligand layer having a ligand that is sensitive to binding with a target analyte. The ligand layer has a ligand layer thickness. In addition, the nanostructure exhibits a decay length and a sensitivity value and the decay length corresponds to the ligand layer thickness and the sensitivity value is not less than about 175 nm per reflective index unit (nm/RIU).

For example, in one embodiment the decay length may be not less than about 0.5 times the ligand layer thickness and not greater than about 1.5 times the ligand layer thickness. For example, in one embodiment, the decay length is not less than about 5 nm and not greater than about 18 nm. In another embodiment, the decay length is about 11 nm and the sensitivity value is about 300 nm/RIU.

More generally, the sensitivity value may be maximized for the given ligand layer thickness that corresponds to the decay length. For instance, the decay length may be matched to the size of the ligand.

In the medium of the first aspect, the ligand may be operative to bind with a target analyte within the decay length relative to the sensor media to produce a shift in an optical signal of the sensor media corresponding to the sensitivity value. In one embodiment, the ligand is much larger than the target analyte. For instance, the ligand may be at least one order of magnitude larger than the target analyte. In various embodiments, the ligand, the analyte, or both may comprise a protein, which may be different in the case in which both the ligand and analyte are proteins. In one embodiment the ligand is of a size not less than about 10 kDa and the analyte is of a size not greater than about 1 kDA.

A second aspect also includes a surface plasmon resonance (SPR) sensor medium. The medium of the second aspect may include a nanostructure comprising a ligand layer having a ligand that is sensitive to binding with a target analyte. The nanostructure exhibits a decay length that corresponds to the thickness of a three dimensional surface matrix chemistry containing the ligand layer, as illustrated in FIGS. 5A and 5B..

For instance, the three dimensional surface chemistry may include at least one of dextran, chitosan, polyelectrolyte, or a sugar. Where the three dimensional surface chemistry includes dextran chains, the dextran chains may include a plurality of binding sites to which ligand may be bound in the sensor media. In any regard, the three dimensional surface chemistries may increase the density of analyte binding sites relative to a native or a substantially planar nanostructure surface.

In embodiments of the second aspect, the ligand may also be much larger than the target analyte. For instance, the ligand may be at least one order of magnitude larger than the target analyte. In one embodiment the ligand may be of a size not less than about 10 kDa and the analyte may be of a size not greater than about 1 kDA.

A third aspect includes a method for detection of an analyte in a fluid using a surface plasmon resonance (SPR) sensor. The method includes providing a SPR sensor medium comprising a nanostructure portion comprising a ligand layer having a ligand that is sensitive to binding with a target analyte. The ligand layer has a ligand layer thickness, and the nanostructure exhibits a decay length and a sensitivity value. The decay length corresponds to the ligand layer thickness and the sensitivity value is not less than about 175 nm per reflective index unit (nm/RIU). The method also includes contacting a fluid comprising an analyte with the SPR sensor medium and measuring an optical signal to detect a change in the optical signal in response to the contacting event to measure the analyte in the fluid.

A fourth aspect includes a method for detection of an analyte in a fluid using a surface plasmon resonance (SPR) sensor. The method includes providing a SPR sensor medium comprising a nanostructure comprising a ligand layer having a ligand that is sensitive to binding with a target analyte. The nanostructure exhibts a decay length that corresponds to the thickness of the three dimensional surface matrix chemistry containing the ligand layer. The method also includes contacting a fluid comprising an analyte with the SPR sensor medium and measuring an optical signal to detect a change in the optical signal in response to the contacting to measure the analyte in the fluid.

It will be appreciated by one of skill in the art that a number of feature refinements and additional features are applicable to the various aspects of the invention. These feature refinements and additional features may be used individually or in any combination. As such, each of the features may be, but are not required to be, used with any other feature or combination of features of the various aspects of the invention.

### BRIEF DESCRIPTION OF DRAWINGS

Having thus described the presently disclosed subject matter in general terms, reference will now be made to the accompanying Drawings, which are not necessarily drawn to scale, and wherein:

FIG. 1 shows an example of a plot of the detection signal of carbonic anhydrase II (CAII) on Particle 1;

FIG. 2 shows an example of a plot of the detection signal of CAII on Particle 2;

FIG. 3 shows an example of a plot of the detection signal of Protein A on Particle 1;

FIG. 4 shows an example of a plot of the detection signal of Protein A on Particle 2.

FIG. 5A illustrates the decay length corresponding to the ligand layer thickness for a single layer ligand, and FIG. 5B illustrates the decay length corresponding to the ligand layer for a 3D matrixed ligand layer. The decay length is the electromagnetic field decay length of the nanomaterial.

### DETAILED DESCRIPTION

The presently disclosed subject matter now will be described more fully hereinafter with reference to the accompanying Drawings, in which illustrative embodiments of the presently disclosed subject matter are shown. Like numbers refer to like elements throughout. It is not intended that the embodiments described herein limit the scope of the invention(s) disclosed herein and are provided for illustrative purposes only. That is, the presently disclosed subject matter may be embodied in many different forms and should not be construed as limited to the embodiments set forth herein.

In some embodiments, the presently disclosed subject matter may provide nanostructures for improved molecular detection. Namely, the presently disclosed nanostructures may be used to increase the ligand immobilization shift thereby increasing the shift of the analyte molecule, particularly for small molecule analytes. In one embodiment, nanostructures are provided that have small decay lengths (i.e., decay length is the electromagnetic field decay length of the nanomaterial used to form the nanostructure) and high sensitivity. In another embodiment, nanostructures are provided that have large decay lengths and 3D surface matrix chemistries. Further, the embodiments are applicable to other types of molecules as well. In at least some embodiments, the nanostructures described may be provided in the context of an LSPR sensor such as that described in U.S. Pat. No. 9,322,823 and/or U.S. Pat. Pub. No. 2016/0299134, both of which are incorporated by reference herein in their entirety.

Any metal nanomaterials exhibiting LSPR properties can be used as the nanomaterial. Examples of materials useful for forming the nanostructures of the invention include gold, silver, platinum, gold coated silver, silver coated gold, combinations of these metals, and others. One of skill in the art will recognize a variety of techniques for varying the decay length, such as selection of the nanomaterial composition, size of the nanomaterial, and surface topography of the nanomaterial.

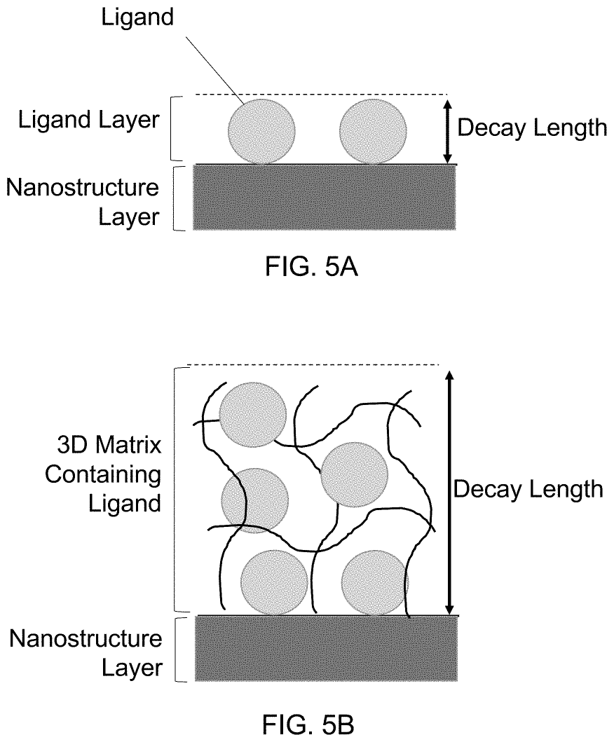

In a first embodiment, nanostructures may be provided that have small decay lengths and high sensitivity (e.g., LSPR sensitivity), as illustrated in FIG. 5A. Small molecules do not typically bind a substantial distance outside of the ligand. This means that the decay length of the nanostructure can be made very small without sacrificing the ability of the nanostructure to detect the small molecule as it will not fall outside of the detection region. Using a decay length that is on the order of the thickness of the ligand layer will increase (e.g., maximize) the shift in the signal of the ligand layer, while still allowing the small molecule to bind within the most sensitive region of the sensing field. This may consequently increase (e.g., maximize) the amount of analyte shift resulting from binding of the analyte with the ligand. Although using the smallest decay length as possible may result in an even larger shift from the ligand, there is risk that the shift from the small molecule will be reduced if it is binding very far away from the decay length. Accordingly, the decay length will typically not be less than about 0.5 times the thickness of the ligand layer and not greater than about 1.5 times the thickness of the ligand layer. In at least some approaches the decay length may be not less than about 5 nm and not greater than about 18 nm. In addition, the sensitivity may be not less than about 175 nm/RIU.

The above assumes that the only parameter that is controlled is the decay length, but in reality changing the decay length will typically also cause the sensitivity (e.g., the LSPR sensitivity or m) to change. The decay length and sensitivity can be modified by changing the nanostructure size, shape, and material. To increase (e.g., maximize) the ligand response (and hence the analyte response) the decay length may be matched to be the size of the ligand while maintaining the highest possible refractive index sensitivity.

A number of examples are provided that demonstrate the increase in the optical signal shift based on use of nanostructures with a small decay length and high sensitivity. In the examples described below, Particle 1 corresponds to a baseline approach in which the nanostructure has both moderate decay lengths and sensitivity. For instance, Particle 1 may have a decay length of about 21 nm and a sensitivity of 150 nm/RIU. In contrast, Particle 2 may have a sensitivity of about 300 nm/RIU and a decay length of 11 nm. In this regard, Particle 2 may correspond to a nanostructure having a short decay length and high sensitivity according to the first embodiment.

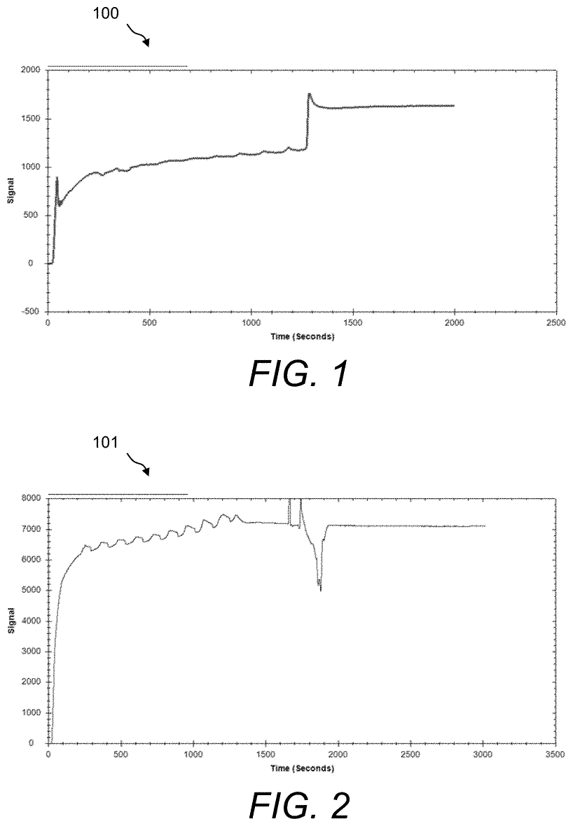

Referring now to plot 100 and plot 101 in FIG. 1 and FIG. 2, the detection signal of CAII immobilization onto nanostructures comprising Particle 1 is shown in FIG. 1 and Particle 2 is shown in FIG. 2. Immobilization was performed via a carboxyl (COOH) functional surface on the nanostructures.

The ligand shift for CAII from Particle 1 is approximately 1600 pm (as best seen in plot 100), while on Particle 2 the ligand shift is 7100 pm (as best seen plot 101). This is a 4.4 times increase in ligand signal which directly translates into a max analyte response that is also 4.4 times higher. Thus, the use of Particle 2 with a high sensitivity and short decay length may be much more sensitive, thus resulting in improved measurement capability using Particle 2.

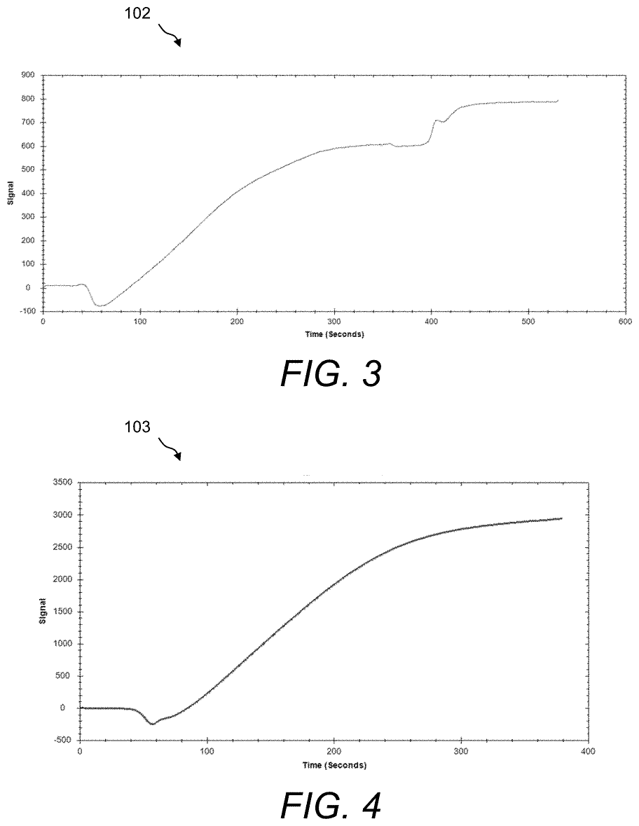

Referring now to plot 102 and plot 103 in FIG. 3 and FIG. 4, the detection signal of Protein A immobilization onto Particle 1 is shown in FIG. 3 and Particle 2 is shown in FIG. 4. Immobilization was done via a carboxyl (COOH) functional surface on the nanostructures. The ligand shift of Protein A on Particle 1 is approximately 800 pm while on Particle 2 it is 3000 pm, a 3.75 times increase. Again, Particle 2 with a high sensitivity and short decay length shows increased sensitivity, thus improving the measurement capability of the system.

A second embodiment includes nanostructures that have large decay lengths (i.e., decay length is the electromagnetic field decay length of the nanomaterial used to form the nanostructure) and 3D surface matrix chemistries, as illustrated in FIG. 5B. Another method to increase the analyte response may be through increasing the density of ligand binding sites on the nanostructure. This can be done by using nanostructures with large decay lengths and a three-dimensional (3D) surface chemistry rather than a planar two-dimensional surface chemistry. The 3D surface chemistry could comprise dextran, chitosan, polyelectrolytes, sugars, and/or other 3D surface chemistries. For example, dextran can be coupled to the nanostructured surface and a large density of binding sites could be present on the dextran chains. This may allow a much larger ligand binding density to be obtained, which would increase the ligand response and increase the maximum analyte response.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A surface plasmon resonance (SPR) sensor medium, comprising:
a nanostructure portion comprising a ligand layer having a ligand that is sensitive to binding with a target analyte, the ligand layer comprising a ligand layer thickness;
wherein the nanostructure comprises a decay length and a sensitivity value; and
wherein the decay length corresponds to the ligand layer thickness and the sensitivity value is not less than about 175 nm per reflective index unit (nm/RIU).

2. The sensor medium of claim 1, wherein the decay length is not less than about 0.5 times the ligand layer thickness and not greater than about 1.5 times the ligand layer thickness.

3. The sensor medium of claim 1, wherein the decay length is not less than about 5 nm and not greater than about 18 nm.

4. The sensor medium of claim 1, wherein the decay length is about 11 nm and the sensitivity value is about 300 nm/RIU.

5. The sensor medium of claim 1, wherein the sensitivity value is maximized for the given ligand layer thickens that corresponds to the decay length.

6. The sensor medium of claim 1, wherein the decay length is matched to a size of the ligand.

7. The sensor medium of claim 1, wherein the ligand is operative to bind with a target analyte within the decay length relative to the sensor media to produce a shift in an optical signal of the sensor media corresponding to the sensitivity value.

8. The sensor medium of claim 1, wherein the ligand is much larger than the target analyte.

9. The sensor medium of claim 8, wherein the ligand is at least one order of magnitude larger than the target analyte.

10. The sensor medium of claim 1, wherein the ligand comprises a first protein.

11. The sensor medium of claim 1, wherein the target analyte comprises a second protein.

12. The sensor medium of claim 1, wherein the ligand is of a size not less than about 10 kDa and the analyte is of a size not greater than about 1 kDa.

13. A surface plasmon resonance (SPR) sensor medium, comprising:
a nanostructure comprising a ligand layer having a ligand that is sensitive to binding with a target analyte;
wherein the nanostructure comprises a decay length that corresponds to the thickness of a three dimensional surface matrix chemistry containing the ligand layer.

14. The sensor medium of claim 13, wherein the three dimensional surface chemistry comprises at least one of dextran, chitosan, polyelectrolyte, or a sugar.

15. The sensor medium of claim 13, wherein the three dimensional surface chemistry comprises dextran chains, wherein the dextran chains comprise a plurality of bindings sites to which ligand may be bound in the sensor media.

16. The sensor medium of claim 13, wherein the ligand is much larger than the target analyte.

17. The sensor medium of claim 16, wherein the ligand is at least one order of magnitude larger than the target analyte.

18. The sensor medium of claim 13, wherein the ligand is of a size not less than about 10 kDa and the analyte is of a size not greater than about 1 Da.

19. The sensor medium of claim 13, wherein the three dimensional surface chemistries increase the density of analyte binding sites relative to a native or a substantially planar nanostructure surface.

20. A method for detection of an analyte in a fluid using a surface plasmon resonance (SPR) sensor, comprising:
providing a SPR sensor medium comprising a nanostructure portion comprising a ligand layer having a ligand that is sensitive to binding with a target analyte, the ligand layer comprising a ligand layer thickness, wherein the nanostructure comprises a decay length and a sensitivity value, and wherein the decay length corresponds to the ligand layer thickness and the sensitivity value is not less than about 175 nm per reflective index unit (nm/RIU);
contacting a fluid comprising an analyte with the SPR sensor medium; and
measuring an optical signal to detect a change in the optical signal in response to the contacting to measure the analyte in the fluid.

21. The method of claim 20, wherein the SPR sensor medium is according to claim 2.

22. A method for detection of an analyte in a fluid using a surface plasmon resonance (SPR) sensor, comprising:
providing a SPR sensor medium comprising a nanostructure comprising a ligand layer having a ligand that is sensitive to binding with a target analyte, wherein the nanostructure comprises a decay length that corresponds to the thickness of a three dimensional surface matrix chemistry containing the ligand layer;
contacting a fluid comprising an analyte with the SPR sensor medium; and
measuring an optical signal to detect a change in the optical signal in response to the contacting to measure the analyte in the fluid.

23. The method of claim 22, wherein the SPR sensor medium is according to claim 13.
